(19) United States
(12) Patent Application Publication
(10) Pub. No.: US 2023/0180836 A1
(43) Pub. Date: Jun. 15, 2023

(54) VAPORIZATION STATE RECOGNITION METHOD, APPARATUS, ELECTRONIC DEVICE AND STORAGE MEDIUM

(71) Applicant: Shenzhen Moore Vaporization Health & Medical Technology Co., Ltd., Shenzhen (CN)

(72) Inventor: Sheng YANG, Shenzhen (CN)

(21) Appl. No.: 18/076,713

(22) Filed: Dec. 7, 2022

(30) Foreign Application Priority Data
Dec. 10, 2021 (CN) 202111506032.5

(51) Int. Cl. A24F 40/46; A24F 40/50

(52) U.S. Cl. CPC A24F 40/46; A24F 40/50

(57) ABSTRACT

A vaporization state recognition method includes: obtaining a heating rate of an atomizing sheet of a vaporizer; and determining a vaporization state of the vaporizer according to the heating rate. In an embodiment, determining the vaporization state of the vaporizer according to the heating rate includes: obtaining a changing trend of the heating rate in a predetermined vaporization time; determining that the atomizing sheet is abnormal or the vaporizer is in a dry heating state in response to the heating rate being in an increasing trend in the predetermined vaporization time; and determining that the vaporizer enters a stable vaporization interval in the determined vaporization time in response to the heating rate being in a declining trend in the predetermined vaporization time.

### CROSS-REFERENCE TO PRIOR APPLICATION

Priority is claimed to Chinese Patent Application No. 202111506032.5, filed on Dec. 10, 2021, the entire disclosure of which is hereby incorporated by reference herein.

### FIELD

This application relates to the field of electronic vaporization technologies, and in particular, to a vaporization state recognition method, an apparatus, an electronic device and a storage medium.

### BACKGROUND

With the development of medical technologies, a vaporization inhalation therapy appears, and is usually used in the treatment of respiratory diseases. An atomizing sheet using an electronic vaporization device vaporizes medicinal liquid into tiny particles, and the vaporized medicine is inhaled and deposited in the respiratory tract and the lung of a patient through breathing, which can directly reach a lesion, accelerate the action of the medicine, so as to achieve a painless, rapid, and effective treatment.

Dry heating is a common problem during using the electronic vaporization device. Long-term dry heating damages the atomizing sheet, resulting in a decrease in performance, a reduction in a vaporization amount, and even a loss of a vaporization function for the atomizing sheet.

### SUMMARY

In an embodiment, the present invention provides a vaporization state recognition method, comprising: obtaining a heating rate of an atomizing sheet of a vaporizer; and determining a vaporization state of the vaporizer according to the heating rate.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a schematic flowchart of a first embodiment of a vaporization state recognition method according to this application;

FIG. 2 is a schematic flowchart of step S2 of the vaporization state recognition method according to FIG. 1;

FIG. 3 is a temperature rise curve diagram of an atomizing sheet in normal vaporization according to this application;

FIG. 4 is a schematic flowchart of step S2 of a second embodiment of the vaporization state recognition method according to this application;

FIG. 5 is a temperature rise curve comparison diagram of the atomizing sheet in different vaporization states according to this application;

FIG. 6 is a schematic flowchart of a third embodiment of the vaporization state recognition method according to this application;

FIG. 7 is a schematic structural diagram of an embodiment of a vaporization state recognition apparatus according to this application;

FIG. 8 is a schematic structural diagram of an embodiment of an electronic device according to this application;

FIG. 9 is a schematic block diagram of an electronic vaporization device according to an embodiment of this application;

FIG. 10 is a schematic structural diagram of a computer-readable storage medium according to an embodiment of this application.

### DETAILED DESCRIPTION

In an embodiment, the present invention provides a vaporization state recognition method, an apparatus, an electronic device and a storage medium provided by this application, resolve a technical problem about how to detect dry heating of an electronic vaporization device in the prior art.

In an embodiment, the present invention provides a vaporization state recognition method, including:

obtaining a heating rate of an atomizing sheet of a vaporizer; and

determining a vaporization state of the vaporizer according to the heating rate.

The determining a vaporization state of the vaporizer according to the heating rate specifically includes: obtaining a changing trend of the heating rate in a predetermined vaporization time;

determining that the atomizing sheet is abnormal or the vaporizer is in a dry heating state in response to the heating rate being in an increasing trend in the predetermined vaporization time; and

determining that the vaporizer enters a stable vaporization interval in the determined vaporization time in response to the heating rate being in a declining trend in the predetermined vaporization time.

After the determining that the vaporizer enters a stable vaporization interval in the determined vaporization time, the method further includes:

determining whether the heating rate in the stable vaporization interval is greater than or equal to a preset threshold; and

determining that the vaporizer is in a dry heating state in response to the heating rate in the stable vaporization interval being greater than or equal to the preset threshold.

The determining whether the heating rate in the stable vaporization interval is greater than or equal to a preset threshold specifically includes:

determining whether the heating rate in each different sub-interval in the stable vaporization interval is greater than or equal to the preset threshold corresponding to each sub-interval.

The predetermined vaporization time is a time period of vaporization for 4 s to 12 s counted from the start of the vaporizer. The start of the vaporizer here means the time when the vaporizer starts to work.

To resolve the above technical problem, a second technical solution provided by this application is to provide a vaporization state recognition apparatus, including an obtaining module and a processing module; where the obtaining module is configured to obtain a heating rate of an atomizing sheet of a vaporizer; and where the processing module is configured to determine a vaporization state of the vaporizer according to the heating rate.

To resolve the above technical problem, a third technical solution provided by this application is to provide an electronic device, including: a memory and a processor, where the memory stores program instructions, and the processor reads the program instructions from the memory to execute the vaporization state recognition method according to any one of the above.

The electronic device is an electronic vaporization device, where the electronic vaporization device includes the atomizing sheet and a temperature characteristic component, where the temperature characteristic component is connected to the processor and is configured to detect the temperature of the atomizing sheet.

The temperature characteristic component is a positive temperature coefficient thermistor or a negative temperature coefficient thermistor.

To resolve the above technical problem, a fourth technical solution provided by this application is to provide a computer-readable storage medium, storing a program file, where the program file is executable to implement the vaporization state recognition method according to any one of the above.

Beneficial effects of this application: different from the prior art, this application discloses a vaporization state recognition method, an apparatus, an electronic device and a storage medium. The vaporization state recognition method including: obtaining a heating rate of an atomizing sheet of a vaporizer; and determining a vaporization state of the vaporizer according to the heating rate. Therefore, whether the vaporizer is dry heating may be recognized in time and the dry heating of the vaporizer may be dealt with in time. In addition, during recognizing the vaporization state of the vaporizer, there is no need to use a sensor such as a probe to contact medicinal liquid, so that a detection is more accurate.

The technical solutions in embodiments of this application are clearly and completely described below with reference to the accompanying drawings in the embodiments of this application. Apparently, the described embodiments are merely some rather than all of the embodiments of this application. All other embodiments obtained by a person of ordinary skill in the art based on the embodiments of this application without creative efforts shall fall within the protection scope of this application.

In the following description, for the purpose of illustration rather than limitation, specific details such as the specific system structure, interface, and technology are proposed to thoroughly understand this application.

The terms “first”, “second”, and “third” in this application are merely intended for a purpose of description, and shall not be understood as indicating or implying relative significance or implicitly indicating the number of indicated technical features. Therefore, features defining “first”, “second”, and “third” can explicitly or implicitly include at least one of the features. In the description of this application, “a plurality of” means at least two, such as two and three unless it is specifically defined otherwise. All directional indications (for example, upper, lower, left, right, front, and back) in the embodiments of this application are only used for explaining relative position relationships, movement situations, or the like between the various components in a specific posture (as shown in the accompanying drawings). If the specific posture changes, the directional indications change correspondingly. In the embodiments of this application, the terms “include”, “have”, and any variant thereof are intended to cover a non-exclusive inclusion. For example, a process, method, system, product, or device that includes a series of steps or units is not limited to the listed steps or units, but further optionally includes a step or unit that is not listed, or further optionally includes another step or component that is intrinsic to the process, method, product, or device.

“Embodiment” mentioned in this specification means that particular features, structures, or characteristics described with reference to the embodiment may be included in at least one embodiment of this application. The term appearing at different positions of this specification may not refer to the same embodiment or an independent or alternative embodiment that is mutually exclusive with another embodiment. A person skilled in the art explicitly or implicitly understands that the embodiments described in this specification may be combined with other embodiments.

This application is described in detail below with reference to the accompanying drawings and embodiments.

An electronic vaporization device generally arranges a sensor such as a probe in a liquid storage cavity. Through the sensor such as the probe being in contact with a to-be-vaporized substrate and detecting the liquid level of the to-be-vaporized substrate, whether there still exists the to-be-vaporized substrate in the liquid storage cavity is determined, so as to determine whether a vaporizer is in a dry heating state. It can be understood that the inventor believes that the vaporizer of the electronic vaporization device is in a dry heating state, and the possible reasons include: 1. the to-be-vaporized substrate stored in the vaporizer is used up; 2. the to-be-vaporized substrate is poorly liquid-supplied; 3. a driving mode of an atomizing sheet does not match with the state of the existing to-be-vaporized substrate. When the atomizing sheet is in the dry heating state, on the one hand, the atomizing sheet is damaged by dry heating. On the other hand, the temperature of the atomizing sheet may increase in the dry heating state, causing the to-be-vaporized substrate (such as medicinal liquid) to be ineffective. However, currently, the dry heating caused by the poor liquid supply of the to-be-vaporized substrate is not detectable in an existing detection manner. Based on the existing problems of the prior art, this application provides a vaporization state recognition method to detect more accurately whether the vaporization state of the vaporizer is the dry heating.

Referring to FIG. 1, FIG. 1 is a schematic flowchart of a first embodiment of a vaporization state recognition method according to this application.

The vaporization state recognition method according to this application specifically includes the following steps.

S1: Obtain a heating rate of an atomizing sheet of a vaporizer.

Specifically, obtaining the temperature of the atomizing sheet; and obtaining the heating rate of the atomizing sheet according to the temperature of the atomizing sheet and a vaporization time corresponding to the atomizing sheet. The temperature of the atomizing sheet may be detected through arranging a temperature characteristic component on the atomizing sheet. The temperature characteristic component may be a positive temperature coefficient thermistor, a negative temperature coefficient thermistor or the like, as long as the temperature of the atomizing sheet can be detected. It can be understood that the atomizing sheet generally includes a piezoelectric ceramic and a metal substrate. The metal substrate may have TCR (temperature coefficient of resistance) performance, so as to obtain temperature of the metal substrate to obtain the temperature of the atomizing sheet. The obtained temperature of the atomizing sheet is a real-time temperature.

S2: Determine a vaporization state of the vaporizer according to the heating rate.

Specifically, the principle of the determining a vaporization state of the vaporizer according to the heating rate is as follows. The inventor of this application finds that when the atomizing sheet is in the dry heating state, the temperature of the atomizing sheet rises rapidly and the temperature rise rate is much greater than the temperature rise rate in normal vaporization; and in a different temperature section, the temperature rise rate is different when the atomizing sheet is in a dry heating state. Therefore, whether the vaporization state of the vaporizer is dry heating may be determined through the heating rate of the vaporizer.

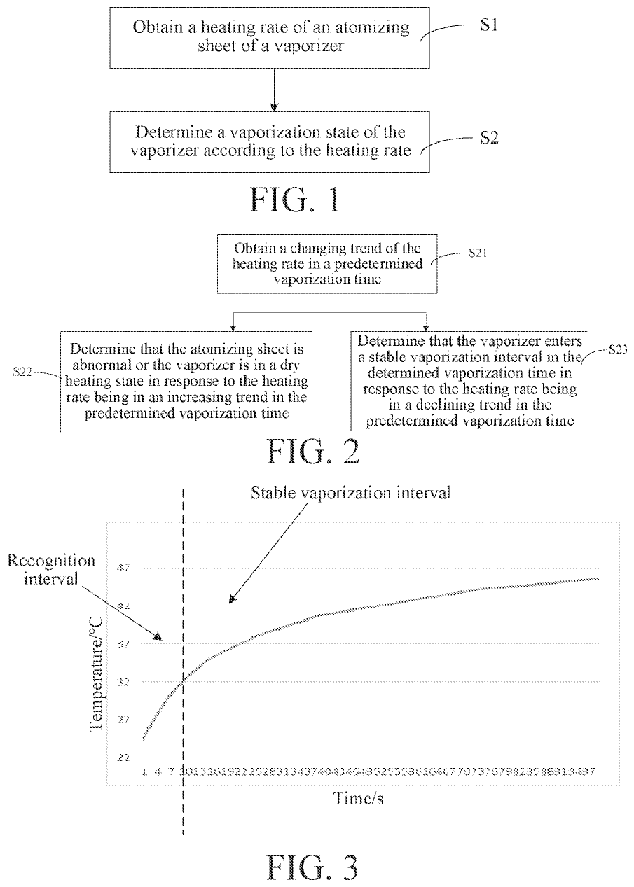

Referring to FIG. 2, FIG. 2 is a schematic flowchart of step S2 of the vaporization state recognition method according to FIG. 1. The determining a vaporization state of the vaporizer according to the heating rate specifically includes the following steps.

S21: Obtain a changing trend of the heating rate in a predetermined vaporization time.

Taking an electronic vaporization device including an atomizing sheet with a microporous thereon and a liquid storage unit as an example, an experiment is carried out. A temperature rise curve of the atomizing sheet during a normal vaporization is obtained through performing a normal vaporization test on the electronic vaporization device. The experiment result is shown in FIG. 3. FIG. 3 is a temperature rise curve diagram of the atomizing sheet in normal vaporization according to this application. In FIG. 3, an X-axis is the number of sampling points, and a Y-axis is the sampling temperature corresponding to the sampling point; in this experiment, a single sampling time interval is 1 s, and a total of 97 sampling points is collected. In FIG. 3, the temperature rise curve of the atomizing sheet in normal vaporization is divided into a recognition interval and a stable vaporization interval.

It can be seen from FIG. 3 that a changing rule of the heating rate in the recognition interval is that: when the electronic vaporization device works, the to-be-vaporized substrate is started to be heated and vaporized. With the progress of the vaporization, a temperature difference appears in the to-be-vaporized substrate. The temperature of a part of the to-be-vaporized substrate which is close to the atomizing sheet, is high. While the temperature of a part of the to-be-vaporized substrate which is away from the atomizing sheet, is low. The to-be-vaporized substrate in a low temperature zone cools down the to-be-vaporized substrate in a high temperature zone, which restricts the heating rate of the to-be-vaporized substrate in the high temperature zone. In this case, the heating rate of the atomizing sheet is relatively high; with the further progress of the vaporization, the temperature of the to-be-vaporized substrate is basically the same and is close to the temperature of the atomizing sheet. The heating rate of the atomizing sheet gradually tends to be gentle, and the vaporization enters the stable vaporization interval. The heating rate of the atomizing sheet gradually tends to be gentle, representing that the changing trend of the heating rate tends to be gentle, that is, the growth of the heating rate is in a declining trend.

It can be seen from FIG. 3 that after the electronic vaporization device starts the vaporization, after around 10 s of the vaporization, the heating rate of the atomizing sheet tends to be gentle, and the electronic vaporization device enters a stable vaporization interval. That is, if the electronic vaporization device is in a normal vaporization state, the electronic vaporization device enters the stable vaporization interval in the predetermined vaporization time (for example, 10 s described above) of vaporization, and the heating rate of the atomizing sheet gradually tends to be gentle. The predetermined vaporization time is the time corresponding to the recognition interval. Therefore, whether the vaporization state is dry heating may be determined through obtaining the changing trend of the heating rate in the predetermined vaporization time. Since a vaporization parameter is arranged different in different electronic vaporization devices, the predetermined vaporization time needed for entering the stable vaporization interval is different. The predetermined vaporization time is arranged to be a time period of vaporization for 4 s to 12 s counted from the start of the vaporizer. The start of the vaporizer here means the time when the vaporizer starts to work.

S22: Determine that the atomizing sheet is abnormal or the vaporizer is in a dry heating state in response to the heating rate being in an increasing trend in the predetermined vaporization time.

After the electronic vaporization device vaporizes for the predetermined vaporization time, the heating rate of the atomizing sheet is still in an increasing trend, representing that the vaporization does not or will not enter the stable vaporization interval. Therefore, the atomizing sheet is abnormal or in a dry heating state. It can be understood that when the atomizing sheet is abnormal, the to-be-vaporized substrate cannot be heated in a predetermined heating manner, and the vaporization cannot enter the stable vaporization interval after vaporizing for the predetermined vaporization time; if there is no to-be-vaporized substrate or there is less to-be-vaporized substrate in a liquid storage cavity when the electronic vaporization device is started, the dry heating appears before entering the stable vaporization interval.

S23: Determine that the vaporizer enters a stable vaporization interval in the determined vaporization time in response to the heating rate being in a declining trend in the predetermined vaporization time.

After the predetermined vaporization time, the heating rate of the atomizing sheet is in a declining trend, representing that the heating rate of the atomizing sheet tends to be gentle, and the vaporization enters a stable vaporization interval. It is the recognition interval before entering the stable vaporization interval, that is recognizing whether the atomizing sheet is abnormal or the atomizing sheet is in the dry heating state when the electronic vaporization device is started.

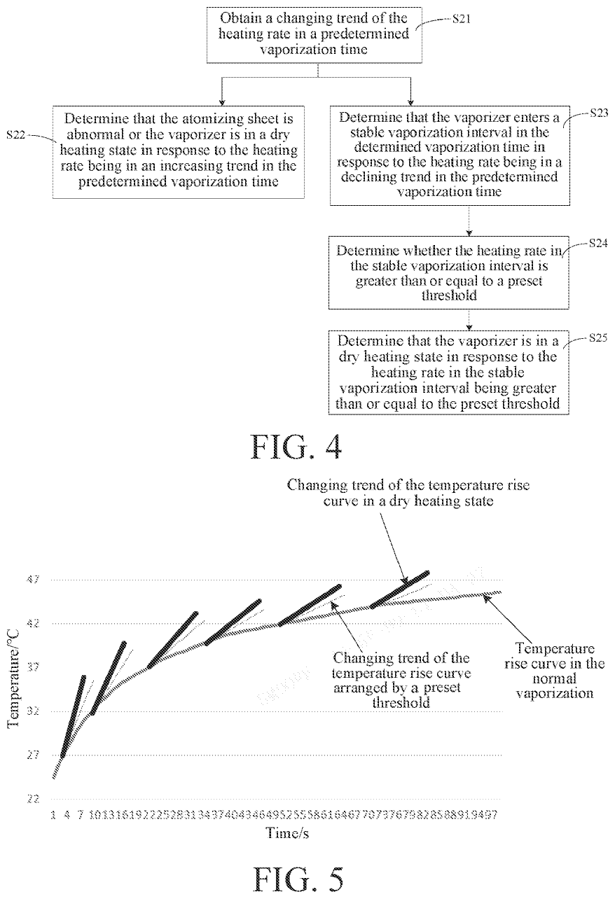

It can be understood that after the vaporization enters the stable vaporization interval, with the progress of the vaporization, the to-be-vaporized substrate is used up and the dry heating phenomenon may appear. Therefore, after entering the stable vaporization interval, the heating rate of the atomizing sheet needed to be detected continuously to determine whether the vaporization state is dry heating. Based on this, this application provides another embodiment of the vaporization state recognition method. Referring to FIG. 4, FIG. 4 is a schematic flowchart of step S2 of a second embodiment of the vaporization state recognition method according to this application.

In the second embodiment of the vaporization state recognition method, step Si and step S2 are the same as the step Si and the step S2 in the first embodiment of the vaporization state recognition method, and details are not described herein again. The difference is that, after step S23, the embodiment further includes:

S24: Determine whether the heating rate in the stable vaporization interval is greater than or equal to a preset threshold. When the atomizing sheet is in the dry heating state, the temperature of the atomizing sheet rises rapidly, that is, whether the vaporization state of the atomizing sheet is dry heating may be determined through arranging the preset threshold and comparing the heating rate of the atomizing sheet with the preset threshold. It can be understood that the real-time heating rate of the atomizing sheet is compared with the preset threshold.

It can be seen from FIG. 3, when the atomizing sheet is in the normal vaporization state, after entering the stable vaporization interval, different heating rates correspond to different temperatures sub-intervals; and in order to accurately detect whether the vaporization state of the atomizing sheet is dry heating, different temperature sub-intervals are arranged with different preset thresholds for comparison. That is, in an embodiment, whether the vaporization state is dry heating may be detected through determining whether the heating rate in each different sub-interval in the stable vaporization interval is greater than or equal to the preset threshold corresponding to each sub-interval.

Specifically, taking an electronic vaporization device including an atomizing sheet with a microporous thereon and a liquid storage unit as an example, an experiment is carried out through performing a normal vaporization test and a dry heating vaporization test on the electronic vaporization device in a constant power. Obtaining the temperature rise curve of the atomizing sheet in the normal vaporization and the changing trend of the temperature rise curve of the atomizing sheet in the state of dry heating, selecting an intermediate value between the heating rate of the normal vaporization and the heating rate of the dry heating as a dry heating recognition preset threshold, and drawing the temperature rise curve in the normal vaporization and the changing trend of the temperature rise curve in the state of dry heating according to the changing trend of the temperature rise curve arranged by the preset threshold to obtain FIG. 5. FIG. 5 is a temperature rise curve comparison diagram of the atomizing sheet in different vaporization states according to this application. In FIG. 5, an X-axis is the number of sampling points, and a Y-axis is the sampling temperature corresponding to the sampling point; in this experiment, a single sampling time interval is 1 s, and a total of 97 sampling points is collected. The test is carried out on the atomizing sheet under a condition of a vaporization temperature range of 20° C.-60° C.

Exemplarily, the stable vaporization interval is divided into four different sub-intervals, such as 30° C.-35° C., 35° C.-40° C., 40° C.-50° C., and 50° C.-60° C. Firstly, obtain the temperature rise curve of the atomizing sheet during the vaporization through the normal vaporization test, and calculate the heating rates of the above four different sub-intervals; further, obtain the heating rates of the atomizing sheet in the above four different sub-intervals during the state of dry heating; then, for one of the sub-intervals, select the intermediate value of the heating rate in the normal vaporization and the heating rate in the state of dry heating as the preset threshold corresponding to the sub-interval. The temperature of the atomizing sheet is 30° C.-35° C., and the corresponding preset threshold of the heating rate is 2° C./S. The temperature of the atomizing sheet is 35° C.-40° C., and the corresponding preset threshold of the heating rate is 1.5° C./S. The temperature of the atomizing sheet is 40° C.-50° C., and the corresponding preset threshold of the heating rate is 1.2° C./S. The temperature of the atomizing sheet is 50° C.-60° C., and the corresponding preset threshold of the heating rate is 1° C./S.

It can be understood that in the above test, after the vaporization for 4 s to 12 s counted from the start of the electronic vaporization device (that is, the time when the electronic vaporization device starts to work), the temperature of the atomizing sheet reaches 30° C., entering the stable vaporization interval at the same time. The division of the different sub-intervals in the stable vaporization interval may be designed according to needs, as long as the preset threshold is greater than the heating rate in the normal vaporization and lesser than the heating rate in the state of dry heating.

S25: Determine that the vaporizer is in a dry heating state in response to the heating rate in the stable vaporization interval being greater than or equal to the preset threshold.

In the stable vaporization interval, the heating rate of the atomizing sheet is greater than or equal to the preset threshold, while the preset threshold is greater than the heating rate in the normal vaporization state, representing that the temperature of the atomizing sheet rises rapidly and the vaporizer is in the dry heating state.

When whether the vaporization state is dry heating may be detected through determining whether the heating rate in each different sub-interval in the stable vaporization interval is greater than or equal to the preset threshold corresponding to each sub-interval, determine that the vaporizer is in the dry heating state, when the real-time heating rate in each different sub-interval is greater than the preset threshold corresponding to each sub-interval.

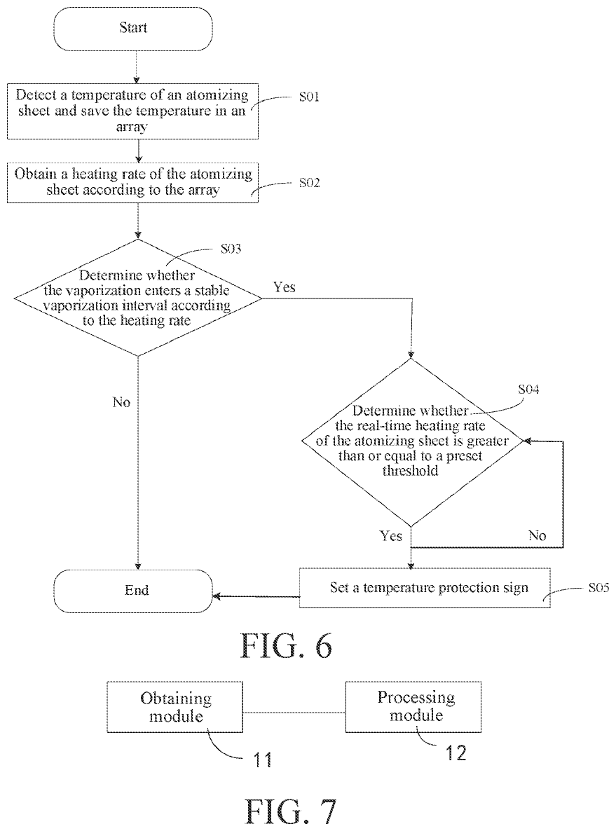

Referring to FIG. 6, FIG. 6 is a schematic flowchart of a third embodiment of the vaporization state recognition method according to this application.

In the third embodiment of the vaporization state recognition method, specifically including:

S01: Detect the temperature of the atomizing sheet and save the temperature in an array.

The temperature of the atomizing sheet may be detected through arranging a temperature characteristic component on the atomizing sheet. The temperature characteristic component may be a positive temperature coefficient thermistor, a negative temperature coefficient thermistor or the like, as long as the temperature of the atomizing sheet can be detected. The temperature of the atomizing sheet may also be obtained through the TCR performance of the atomizing sheet.

S02: Obtain a heating rate of the atomizing sheet according to the array.

The heating rate of the atomizing sheet is obtained through calculating the temperature of the atomizing sheet in the array and the corresponding vaporization time.

S03: Determine whether the vaporization enters the stable vaporization interval according to the heating rate.

When the electronic vaporization device starts for the predetermined vaporization time, and the heating rate is in a declining trend, determine that the vaporization enters the stable vaporization interval and perform step S04; when the electronic vaporization device starts for the predetermined vaporization time, and the heating rate is in an increasing trend, determine that the vaporization state of the vaporizer is dry heating or the atomizing sheet is abnormal.

S04: Determine whether the real-time heating rate of the atomizing sheet is greater than or equal to a preset threshold.

If yes, perform step S05; if no, continue to determine whether the real-time heating rate is greater than the preset threshold.

In the stable vaporization interval, the heating rate of the atomizing sheet is greater than or equal to the preset threshold, while the preset threshold is greater than the heating rate in the normal vaporization state, representing that the temperature of the atomizing sheet rises rapidly and the vaporizer is in the dry heating state.

When whether the vaporization state is dry heating may be detected through determining whether the heating rate in each different sub-interval in the stable vaporization interval is greater than or equal to the preset threshold corresponding to each sub-interval, determine that the vaporizer is in the dry heating state, when the real-time heating rate in each different sub-interval is greater than the preset threshold corresponding to each sub-interval.

S05: Set a temperature protection sign.

The temperature protection sign is set when the atomizing sheet is determined to be in the dry heating state, for example, stopping the vaporizer, so as to prevent a long-term dry heating from damaging the atomizing sheet which results in a decrease of the performance of the atomizing sheet, thereby affecting the performance of the electronic vaporization device.

Referring to FIG. 7, FIG. 7 is a schematic structural diagram of an embodiment of a vaporization state recognition apparatus according to this application.

The vaporization state recognition apparatus includes an obtaining module 11 and a processing module 12; the obtaining module 11 is configured to obtain a heating rate of an atomizing sheet of a vaporizer, and the processing module 12 is configured to determine a vaporization state of the vaporizer according to the heating rate. The vaporization state recognition apparatus may be configured to implement the vaporization state recognition method according to any one of the above, and implement an accurate detection on the vaporization state of the vaporizer.

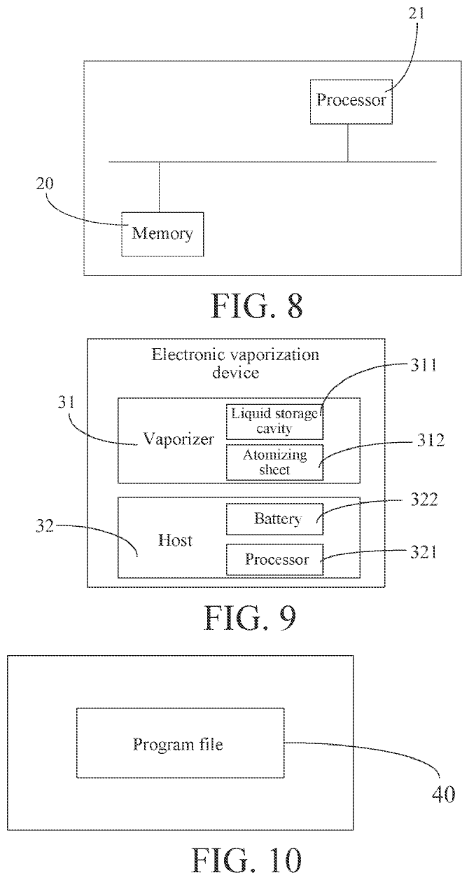

Referring to FIG. 8, FIG. 8 is a schematic structural diagram of an embodiment of an electronic device according to this application. The electronic device includes a memory 20 and a processor 21 connected to each other.

The memory 20 is configured to store and implement program instructions of the vaporization state recognition method according to any one of the above.

The processor 21 is configured to execute the program instructions stored in the memory 20; that is, the processor 21 reads the program instructions stored in the memory 20 from the memory 20 to execute the vaporization state recognition method according to any one of the above.

The processor 21 may be referred to as a CPU (Central Processing Unit). The processor 21 may be an integrated circuit chip with a signal processing capability. The processor 21 may further be a general-purpose processor, a digital signal processor (DSP), an application specific integrated circuit (ASIC), a field programmable gate array (FPGA) or other programmable logic devices, a discrete gate or a transistor logic device, a discrete hardware component. The general-purpose processor may be a microprocessor, or may be any conventional processor, or the like.

The memory 20 may be a memory stick, a TF card or the like, and may store all information in the electronic device of the device, including input original data, a computer program, an intermediate running result and a final running result, all stored in the memory. The memory 20 stores and reads the information according to a position determined by a controller. With the memory 20, the electronic device has a memory function, so as to ensure a normal operation. The memory 20 of the electronic device is divided into a main memory (internal memory) and an auxiliary memory (external memory) according to the purpose, and there is also a classification method dividing the memory 20 into an external memory and an internal memory. The external memory usually is a magnetic medium or an optical disk or the like, which can store information for a long time. The internal memory refers to the storage component on a mainboard, configured to store data and programs being executed currently, but merely configured to store the programs and the data temporarily. The data is lost when the power is turned off or there is a power failure.

In the several embodiments provided in this application, it should be understood that the disclosed method and apparatus may be implemented in other manners. For example, the described apparatus embodiment is merely exemplary. For example, the division of modules or units is merely a logical function division and may be other division during actual implementation. For example, a plurality of units or components may be combined or integrated into another system, or some features may be ignored or not performed. In addition, the displayed or discussed mutual couplings or direct couplings or communication connections may be implemented by using some interfaces. The indirect couplings or communication connections between the apparatuses or units may be implemented in electric, mechanical, or other forms.

The units described as stand-alone components above may be separated physically or not. The components illustrated as units may be physical units or not, and specifically, may be located in one place, or distributed on a plurality of network elements. Some or all of the units may be selected according to actual requirements to achieve the objectives of the solutions of the implementations.

In addition, functional units in the embodiments of this application may be integrated into one processing unit, or each of the units may exist alone physically, or two or more units may be integrated into one unit. The above integrated unit may be implemented in a form of a hardware, or may be implemented in the form of a software functional unit.

When the integrated unit is implemented in the form of a software functional unit and sold or used as an independent product, the integrated unit may be stored in a computer-readable storage medium. Based on such an understanding, the technical solutions of this application essentially, or the part contributing to the related art, or all or some of the technical solutions may be implemented in the form of a software product. The computer software product is stored in a storage medium and includes several instructions for instructing a computer device (which may be a personal computer, a system server, a network device, or the like) or a processor to perform all or some of the steps of the methods in the implementations of this application.

In an embodiment, the electronic device is an electronic vaporization device. The electronic vaporization device is taken as an example for describing the structure of the electronic device in detail. Referring to FIG. 9, FIG. 9 is a schematic block diagram of the electronic vaporization device according to an embodiment of this application.

The electronic vaporization device includes a vaporizer 31 and a host 32. The vaporizer 31 and the host 32 may be detachably connected, or may be integrally formed. The specific may be selected according to needs.

The vaporizer 31 includes a liquid storage cavity 311 and an atomizing sheet 312. The liquid storage cavity 311 is configured to store a to-be-vaporized substrate and the atomizing sheet 312 is configured to vaporize the to-be-vaporized substrate. The host 32 includes a processor 321 and a battery 322. The battery 322 provides power for the atomizing sheet 312, and the processor 321 controls whether the battery 322 provides power for the atomizing sheet 312.

In an embodiment, the vaporizer 31 further includes a temperature characteristic component arranged on the atomizing sheet 312. The temperature characteristic component is connected to the processor 321 and is configured to detect the temperature of the atomizing sheet 312. The temperature characteristic component may be a positive temperature coefficient thermistor, a negative temperature coefficient thermistor or the like, as long as the temperature of the atomizing sheet can be detected.

In another embodiment, the atomizing sheet 312 includes a piezoelectric ceramic and a metal substrate. The metal substrate has TCR performance, so as to obtain the temperature of the metal substrate to obtain the temperature of the atomizing sheet 312.

The electronic vaporization device may be used for vaporization of liquid substrates such as e-liquid and medicinal liquid. It can be understood that the to-be-vaporized substrate stored in the liquid storage cavity 311 may be the liquid substrates such as e-liquid and medicinal liquid, which is selected according to needs.

Referring to FIG. 10, FIG. 10 is a schematic structural diagram of a computer-readable storage medium according to an embodiment of this application. The storage medium in this application stores a program file 40 which can implement all of the above vaporization state recognition method. The program file 40 may be stored in a storage medium in a form of a computer software product and includes several instructions for instructing a computer device (which may be a personal computer, a server, a network device, or the like) or a processor to perform all or some of the steps of the methods described in the implementations of this application. The foregoing storage electronic includes: any medium that can store program code, such as a USB flash disk, a removable hard disk, a read-only memory (ROM), a random access memory (RAM), a magnetic disk or an optical disc, or a terminal device such as a computer, a server, a mobile phone, or a tablet.

The foregoing descriptions are merely implementations of this application, and the patent scope of this application is not limited thereto. All equivalent structure or process changes made according to the content of this specification and accompanying drawings in this application or by directly or indirectly applying this application in other related technical fields shall fall within the protection scope of this application.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. A vaporization state recognition method, comprising:
obtaining a heating rate of an atomizing sheet of a vaporizer; and
determining a vaporization state of the vaporizer according to the heating rate.

2. The method of claim 1, wherein determining the vaporization state of the vaporizer according to the heating rate comprises:
obtaining a changing trend of the heating rate in a predetermined vaporization time;
determining that the atomizing sheet is abnormal or the vaporizer is in a dry heating state in response to the heating rate being in an increasing trend in the predetermined vaporization time; and
determining that the vaporizer enters a stable vaporization interval in the determined vaporization time in response to the heating rate being in a declining trend in the predetermined vaporization time.

3. The method of claim 2, wherein, after the determining that the vaporizer enters a stable vaporization interval in the determined vaporization time, the method further comprises:
determining whether the heating rate in the stable vaporization interval is greater than or equal to a preset threshold; and
determining that the vaporizer is in a dry heating state in response to the heating rate in the stable vaporization interval being greater than or equal to the preset threshold.

4. The method of claim 3, wherein determining whether the heating rate in the stable vaporization interval is greater than or equal to a preset threshold comprises:
determining whether the heating rate in each different sub-interval in the stable vaporization interval is greater than or equal to the preset threshold corresponding to each sub-interval.

5. The method of claim 2, wherein the predetermined vaporization time is a time period of vaporization for 4 seconds to 12 seconds counted from the start of the vaporizer.

6. A vaporization state recognition apparatus, comprising:
an obtaining module configured to obtain a heating rate of an atomizing sheet of a vaporizer; and
a processing module configured to determine a vaporization state of the vaporizer according to the heating rate.

7. An electronic device, comprising:
a memory; and
a processor,
wherein the memory stores program instructions, and
wherein the processor is configured to read the program instructions from the memory to execute the vaporization state recognition method of claim 1.

8. The electronic device of claim 7, wherein the electronic device comprises an electronic vaporization device,
wherein the electronic vaporization device comprises the atomizing sheet and a temperature characteristic component, and
wherein the temperature characteristic component is connected to the processor and is configured to detect a temperature of the atomizing sheet.

9. The electronic device of claim 8, wherein the temperature characteristic component comprises a positive temperature coefficient thermistor or a negative temperature coefficient thermistor.

10. One or more non-transitory computer-readable mediums having processor-executable instructions stored thereon, wherein the processor-executable instructions, when executed, facilitate the vaporization state recognition method of claim 1.
